(19) United States
(12) Patent Application Publication
(10) Pub. No.: US 2017/0067019 A1
(43) Pub. Date: Mar. 9, 2017

(54) Method of Continuous Mass Production of Progenitor Stem-like Cells Using a Bioreactor System

(71) Applicant: BioReactor Sciences LLC, Lawrenceville, GA (US)

(72) Inventor: Timothy Ray Ho, Atlanta, GA (US)

(21) Appl. No.: 15/140,578

(22) Filed: Apr. 28, 2016

Related U.S. Application Data
(60) Provisional application No. 62/215,111, filed on Sep. 7, 2015.

(51) Int. Cl. C12N 5/071; C12Q 3/00

(52) U.S. Cl. CPC C12N 5/063; C12Q 3/00; C12N 2513/00; C12N 2511/00

(57) ABSTRACT

Disclosed herein is a method of culturing cells for cell therapy in a bioreactor.

This application claims the benefit of U.S. provisional application No. 62/215,111, filed on Sep. 7, 2015 and U.S. provisional application No. 62/286,973, filed on Jan. 26, 2016, which are incorporated herein by reference in their entirety.

### BACKGROUND

Keratinocytes derived from epidermis, oral mucosa and urothelium are used in the construction of cell based tissue engineering and regenerative medicine applications. Several methods are being developed to obtain cells with functional plasticity to construct artificial tissue for transplantation, to correct specific systemic diseases and as a source for cell-mediated wound healing therapies. But a method to grow adult somatic cells with maximum plasticity, from human tissue, that circumvents many of the well-known and currently debated ethical and scientific problems associated with use of embryonic derived stem cells or induced pluripotent stem cells, has not yet been developed. Traditional monolayer culture techniques utilizing trypsin for harvesting the cells results in small quantities of cells and as the cells from each monolayer are expanded by passage the ability of the daughter cells to divide is diminished (Hayflick phenomenon). Also, traditional monolayer culture techniques have several risks such as low efficiency of operations since the process is highly dependent upon manual labor; contamination of the culture and deleterious drift (genotypic or phenotypic) possibly due to the changing environmental conditions resulting from traditional manual culture techniques. Marcelo et al 2012 has demonstrated a process which eliminates these risks demonstrating that human epithelial keratinocytes in primary culture can be induced by tissue culture manipulation to produce, without the use of enzymes for passaging, large numbers of small cells in a combined suspension/monolayer culture using traditional culture technique with regular T-flasks. They refer to the small cells as e-PUK (epithelial Pop-Up Keratinocyte) cells. This method significantly improves the production of keratinocyte cells without damage of enzymatic treatment and also enhances production efficiency. The traditional culture technique however only allows the production of e-PUKs from the first passage of keratinocyte monolayer for 7 or less days and requires generating another monolayer from the e-PUK generated from the first passage of cells to continue the subsequent production of e-PUK. The life of the subsequent monolayers and number of passages get shorter and burns out within few passages of monolayer cells due to the lack of ability to properly optimize the cell growing condition using these traditional techniques. Additionally these traditional techniques require substantial labor and manual operation in an open system which is subject to greater risk of contamination.

### SUMMARY

In one aspect, disclosed here are methods of continuous mass production of progenitor stem-like cells using a bioreactor system without requirement of enzyme digestion for subculturing, and from one single monolayer cells, said method comprising of seeding a bioreactor with primary cells derived from the tissue obtained from a biopsy, a cell bank of primary cells, or progenitor cells of said primary cells directly from another seed bioreactor; incubating attached cells under controlled conditions to form a monolayer of parent cells; culturing in a semi-continuous or continuous mode; wherein during the culturing the nutrient and oxygen tension maintain at condition that enable the life of the monolayer cells to continually proliferate and produce the pop-up progenitor cells and subsequently the primary cells for a greatly expanded time; wherein enzyme digestion is not used to facilitate subculturing; removing the suspended pop-up progenitor cells along with the medium replacement under controlled timely manner; and harvesting the progenitor pop-up cells directly from the bioreactor for immediate use, cryopreservation, or removal to seed a second larger production bioreactor or to seed a flask for traditional cell production for cell banking.

Also disclosed are methods of any preceding aspect wherein the bioreactor is a closed system bioreactor.

Also disclosed are methods of any preceding aspect, wherein the bioreactor comprises a 2D or 3D carrier.

Also disclosed are methods of any preceding aspect, wherein the monolayer of step b forms on the carrier.

Also disclosed are methods of any preceding aspect, wherein the progenitor/stem-like cells are progenitor cells of keratinocyte, melanocyte, fibroblast, endothelial cell, urethral cell, skin cell, gingival cells, tongue cells, ligament cells, and mesothelial cells.

Also disclosed are methods of any preceding aspect, wherein said bioreactor comprises multiple of two or more openings and peristaltic pumps for introducing or removing liquid with outer containers, or for gas and medium exchange, and a control mechanism.

Also disclosed are methods of any preceding aspect, wherein said control scheme for nutrient replacement of the monolayer cell comprises adjusting the medium replacement frequency (cycle time t3) in semi-continuous mode by the equation:

t3=(C0−Cmin)/(dR+dC2/t2)

where t1 and t2 are the first and second cycle time of the most recent 2 cycles; dC2 is the difference of glucose concentration change during the second of the most recent 2 cycles; C0 is the concentration (mg/dl) of the fresh medium; Cmin is the minimum concentration to be maintained in the culture; dR=dC2/t2−dC1/t1 and is the change of glucose consumption rates between the two previous cycles, cycle 1 & 2, where dC1 is the same as dC2 but for cycle 1. Also disclosed are methods of any preceding aspect, wherein the continuous feeding rate F3 for the next monitoring cycle is calculated by the following equation:

F3=dR/(C0−Cmin)

where dR=dC2/t2−dC1/t1 is the change of glucose consumption rates between the two previous monitoring cycles, cycles 1 & 2, as calculated in the previous semi-continuous mode, then the following cycle times are the maximum permissible time for total medium replacement in the continuous operation mode.

Also disclosed are methods of any preceding aspect, wherein said removal of pop-up progenitor cells in a controlled timely manner to prevent the cells from dying and poisoning the monolayer cells is dependent upon the maximum permissible time that said progenitor cells can remain alive in suspension without attachment, and accomplished by discharging said progenitor cells along with the spent medium during the complete medium replacement in semi-continuous operation mode or intermittent total medium replacement at the maximum permissible time in continuous operation mode.

### BRIEF DESCRIPTION OF THE DRAWINGS

FIG. 1 shows culturing primary adult human epithelial progenitor/“stem-like cells” without requiring enzymatic treatment using a traditional culture technique.

FIG. 2 shows a schematic diagram showing the illustration of continuously producing the stem-like epithelial cells for a greatly expanded time period from the only monolayer cells of the first passage of parent primary cells using an automatic T-75 flask bioreactor system.

FIG. 3 shows a schematic diagram showing how an integrated automatic T-flask bioreactor system being used to carry out the continuous producing method shown in FIG. 2.

FIG. 4 shows a schematic diagram showing the illustration of continuously producing the stem-like epithelial cells for a greatly expanded time period from the only monolayer cells of the production bioreactor utilizing vessels with multiple flat surface plates such as Corning's Hyperflask or Cellstack, ThermoFisher Scientific's Cell Factory or Paul's Xpansion.

### DETAILED DESCRIPTION

Disclosed herein is a method of continuous mass production of progenitor stem-like cells using a bioreactor system without requirement of enzyme digestion for subculturing, and from one single monolayer cells, said method comprising a) seeding a bioreactor with primary cells derived from the tissue obtained from a biopsy, a cell bank of primary cells, or progenitor cells of said primary cells directly from another seed bioreactor; b) incubating attached cells under controlled conditions to form a monolayer of parent cells; c) culturing in a semi-continuous or continuous mode; wherein during the culturing the nutrient and oxygen tension is maintained at a condition to enable the life of the monolayer cells to continually proliferate and produce the pop-up progenitor cells and subsequently the primary cells for a greatly expanded time; wherein enzyme digestion is not used to facilitate subculturing; d)removing the suspended pop-up progenitor cells along with the medium replacement under controlled timely manner; and e) harvesting the progenitor pop-up cells directly from the bioreactor for immediate clinical use, cryopreservation, or removal to seed a second larger production bioreactor or to seed a flask for traditional cell production for cell banking.

As disclosed herein, cells are seeded and cultured to form a monolayer on a bioreactor. As used herein, bioreactor refers to any manufactured or engineered device or system that supports a biologically active environment to grow cells or tissues in the context of cell culture. In contrary to traditional cell culture techniques, a bioreactor can be engineered to manipulate the environmental condition of the vessel in a closed condition for best culturing the cells. The bioreactor can range in any size from a small laboratory scale in milliliters to a large production scale in the tens of thousands of liters.

As disclosed herein the bioreactor is a closed system bioreactor wherein all openings of said bioreactor vessel are connected with tubing or connectors and the liquid inside and outside of said vessel are exchanged through non-invasive valves or pumps during the entire operation so that there is no liquid content ever open to the external environment through human intervention.

As disclosed herein the bioreactor comprises vessel with carriers. Said carriers are 2D smooth surface or 3D fibrous carriers or scaffolds. A 2D carrier such as that its material is made of polystyrene or like material and is a smooth, flat or curved non-porous surface so that the cells can attach and grow only on the surface and to form the monolayer. A 3D carrier such as that made of glass beads, ceramic, polyester, or polyurethane or like materials has a porous surface for cells to attach but only form monolayer. Said both carriers are commonly treated with low temperature corona discharge plasma to change the surface properties to improve adhesion.

As disclosed herein said bioreactor comprises vessel with multiple or 2, 3, 4, 5, 6, 7, 8, 9 or more openings and peristaltic pumps for introducing or removing liquid with outer containers, or for gas exchange and medium exchange, and a control mechanism wherein a computerized controller is performed to control pH, temperature, oxygen tension, sampling, filling, seeding, culturing, gas and medium exchange and harvesting.

As used herein, for a small bioreactor system the seeded primary cells are obtained from the tissue through traditional isolation methods or a cell bank. The seeded cells are attached to the carriers, incubated and cultured in the small bioreactor with optimal temperature, pH and nutrient control by frequent fresh medium exchange as needed until the cells form the monolayer and reach greater than 80% confluency. For a large bioreactor system with high surface area requiring large quantity of seed, the inoculum are obtained as pop-up progenitor cells produced from the smaller seed bioreactor which is a smaller bioreactor seeded with primary cells isolated from tissue or cell bank as described above. Similarly, the seeded cells attached to the carriers of the large bioreactor are incubated and cultured under optimal control of environmental condition as the small bioreactor described above.

As disclosed herein, after the monolayer cells reach greater than 80% confluency the control of fresh medium replacement in a semi-continuous or continuous mode, oxygen tension, production and harvesting of pop-up progenitor cells begin. Under the optimal control of environmental condition, the life of monolayer cells is substantially extended and so does the production of progenitor pop-up cells. Also only the monolayer cells of the bioreactor is used for the production of progenitor pop-up cells throughout the production process. Said semi-continuous mode in the production process is where the e-PUK cells are produced continuously from the monolayer but the cells along with medium are replaced and harvested intermittently and batchwisely.

Under this operation mode the glucose concentration is reduced gradually from high to low before the fresh medium is replaced. For said continuous mode the fresh medium is continuously fed to the system at calculated rate and withdrawn the spent medium at the same rate so that the glucose concentration of the medium in the vessel remains at the constant desirable critical level rather than varying as the semi-continuous mode does.

Firstly, under the closed system of bioreactor, the nutrient control of the culture medium is determined by the consumption rate of a key nutrient component, glucose, in the medium as an indicator. Glucose concentration is measured by a portable glucose meter or an automatic biochemical analyzers of which the measurement includes glucose and many other biochemicals in the culture medium such as pH, Lac, Gln, MH₄, PO₂, PCO₂, Na+, K+, PO₄, Gly, Ca++ are measured simultaneously and any of which can be used as an indicator if warrants. Then the consumption rate and frequency rate are calculated automatically using the following formula and also automatically feedback to the controller for execution.

For semi-continuous mode the frequency (cycle time) of medium replacement t₃ for the next cycle is calculated by the following equation (1):

t₃=(C₀−Cₘᵢₙ)/(dR+dC₂/t₂)

where t₁ and t₂ are the first and second cycle time of the most recent 2 cycles; dC₂ is the difference of glucose concentration change during the second of the most recent 2 cycles; C₀ is the concentration (mg/dl) of the fresh medium; Cₘᵢₙ is the minimum concentration to be maintained in the culture;

dR=dC₂/t₂−dC₁/t₁

and is the change of glucose consumption rates between the two previous cycles, cycle 1 & 2, where dC₁ is the same as dC₂ but for cycle 1.

For continuous mode the continuous feeding rate for the next monitoring cycle is calculated by the following equation (2):

F₃=dR/(C₀−Cₘᵢₙ)

where dR=dC₂/t₂−dC₁/t₁ and is the change of glucose consumption rates between the initial two previous monitoring cycles, cycles 1 & 2, as calculated in the previous semi-continuous mode, subsequently the cycle time is preferably as long as possible but not exceeding the maximum permissible time for total medium replacement to remove the aged pop-up cells. Therefore the cycle times are the maximum permissible time.

Secondly, oxygen tension is controlled by introducing the gas mixture of air, nitrogen and CO₂ gases at the desired oxygen percentage concentration to the bioreactor for culturing the monolayer cells in the bioreactor.

As disclosed herein said removal of pop-up progenitor cells in a controlled timely manner to prevent the cells from dying and poisoning the monolayer cells is dependent upon the maximum permissible time that said progenitor cells can remain alive in suspension without attachment and accomplished by discharging the progenitor cells along with the spent medium during the medium replacement in semi-continuous operation mode or intermittent total medium replacement at the maximum permissible time in continuous operation mode. Said pop-up progenitor cells are relatively small, adherent and fragile. The cells die after long suspension without attachment to a surface and subsequently poison the monolayer cells. The life of pop-up cells in suspension is cell dependent. For keratinocyte progenitor cells the life is about 24 hours. The bioreactor system allows for the removal of the continuously produced pop-up cells away from the monolayer cells in the system along with the total medium replacement for nutrient control as shown above in the semi-continuous operation mode if the cycle time is less than the maximum permissible time (life time). If not, the batch volume of medium is adjusted so that the cycle time would fall within the life time. In the continuous operation mode, the age of cells inside of said bioreactor vessel is in Poisson distribution. The cells with age greater than the life time always exist. Therefore, an intermittent total medium replacement at the maximum permissible time (life time) interval to remove the aged cells is necessary. However, in a continuous mode the cycle time is preferably to be as long as possible but not exceeding the maximum permissible time for total medium replacement Therefore the cycle times are the maximum permissible time.

As disclosed herein, the pop-up progenitor cells produced and removed from the bioreactor are harvested for immediate seeding and forming the soft-tissue for the clinical or research use; or cryopreserved by future clinical use; or seeding to another larger production bioreactor use. The progenitor cells are also however conveniently harvested by letting the cells attach to the surface of another flask or like and to grow to confluency and then be trypsinized, harvested, cryopreserved as traditional culture method for later clinical or research or cell banking use but without requiring the use of traditional enzymatic subculturing as traditional culturing method does.

Furthermore, many analytical instruments can be used to integrate with the bioreactor system to monitor other parameters from the system such as Cell Counter device by Beckman Coulter to measure the cell number, viability and size; or Near Infrared analyzer by LT Industries to monitor medium contents in real time for further system analysis and to provide feedback control and optimization of the process.

As used herein, wherein the progenitor/stem-like cells are progenitor cells of keratinocyte, melanocyte, fibroblast, endothelial cell, urethral cell, skin cell, gingival cells, tongue cells, ligament cells, and mesothelial cells or like. All of these primary cells are adherent cells which require attachment for growth and have the common feature of contact inhibition. The cells inhibit to contact each other and have the tendency to continue to proliferate under proper condition and pop-up or pop out the progenitor cells as the monolayer cells reach greater than 80% confluency.

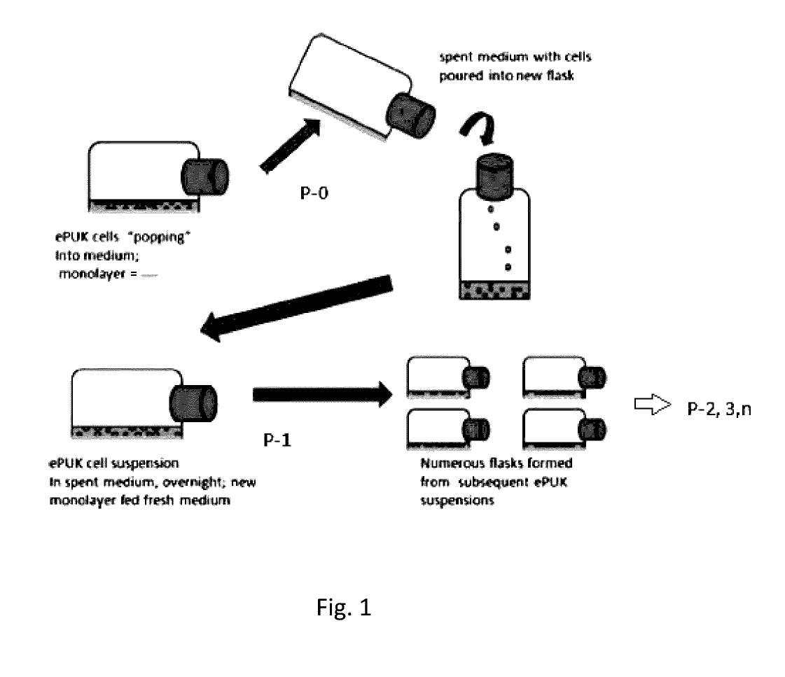

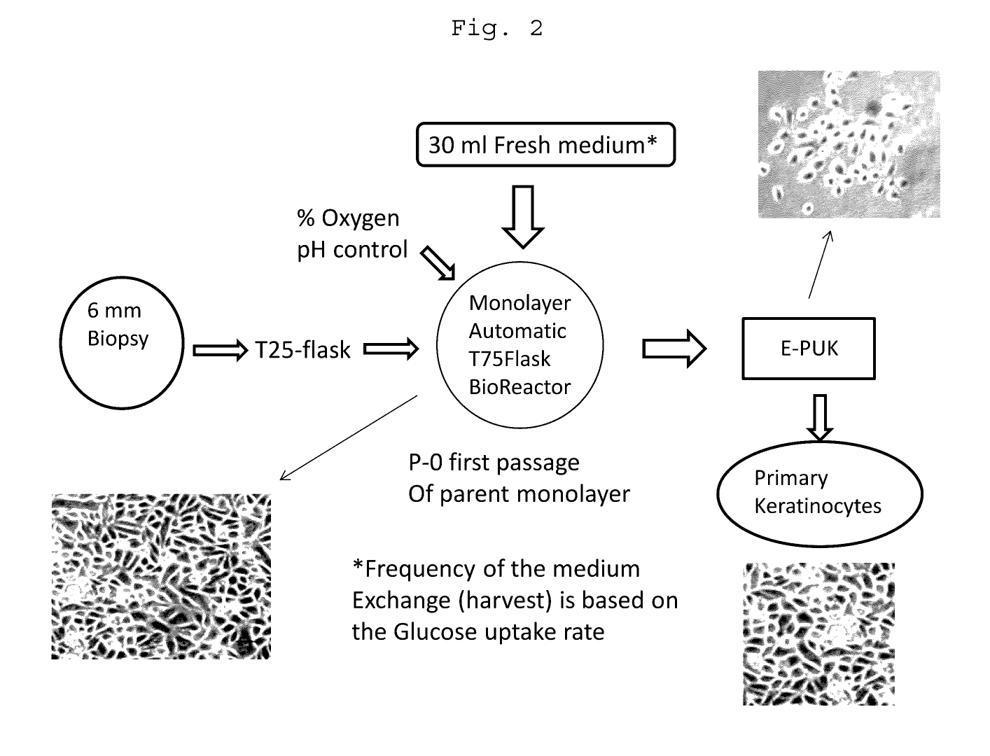

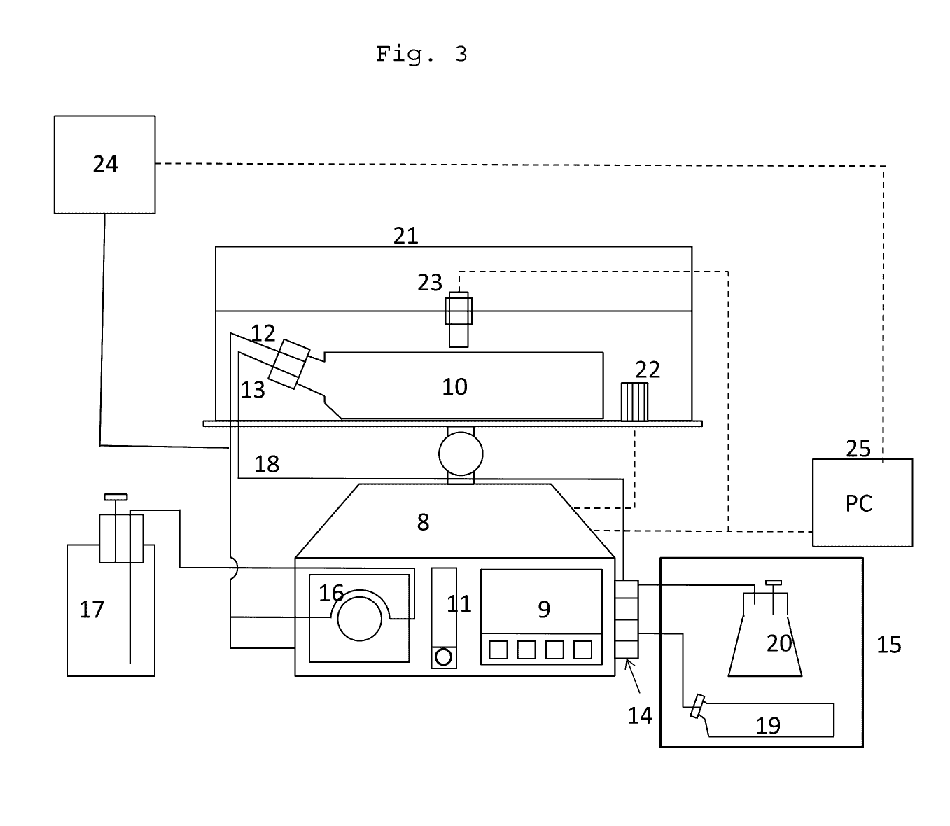

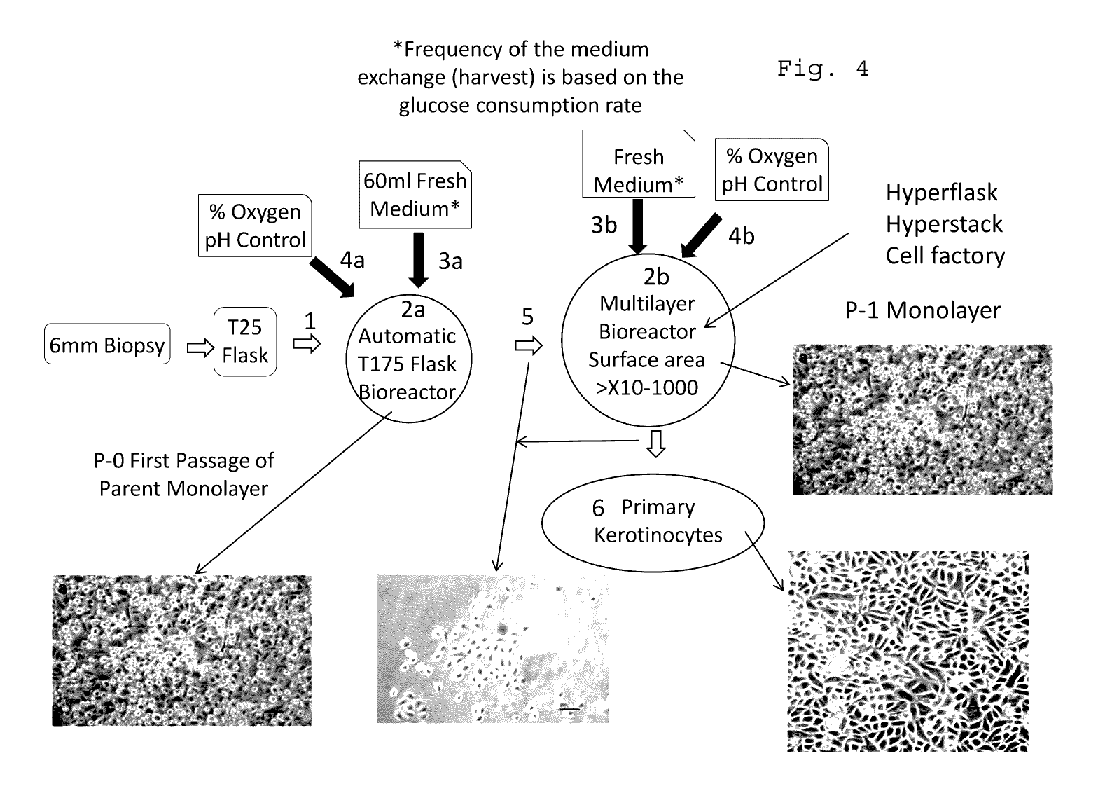

In the following, FIG. 1 shows the scheme of the production method using traditional culture technique; FIG. 2 illustrates a continuous production of epithelial pop-up keratinocyte (e-PUK) cells using an automatic T-flask bioreactor system; FIG. 3 and FIG. 4 display the automatic T-flask bioreactor and the multi-plate production bioreactor to perform this novel continuous production process. This novel production method is applicable to any other bioreactor system which contains 2D or 3D carriers for the cells to attach, grow to form monolayer and produce and release the e-PUK cells or like, and to run under a semi-continuous or continuous mode enabling to maintain optimal environmental control of nutrient condition and oxygen tension throughout the production process; and also effectively removes the e-PUK cells or like before dying and poisoning the culture. For a large production bioreactor a small seed bioreactor is required to produce e-PUK cells or like as inoculum. Both seed and production bioreactors use the same control scheme and only one single monolayer cells of parent primary cells for the entire production of progenitor cells in each bioreactor except the former uses the single passage of parent cells derived from the tissue and the latter uses the e-PUK cells or like produced from the former.

FIG. 1 is a schematic diagram of the protocol of the method developed by Marcelo et al for producing e-PUK cells in a traditional culture technology using conventional T-flasks. This protocol utilizing the traditional culture method illustrates how the cells can be propagated through many passages and many monolayer cells in numerous T-flasks without using trypsin to produce the progenitor e-PUK cells. As shown, they discovered that the monolayer of primary keratinocyte derived from dermis can continue to grow after reaching 100% confluency if 2 folds of original culture medium volume were replaced. They were small floating cells, referred to as e-PUKs, which were attached to a new surface and continued to grow to fill the surface and form the regular cells. However, because of lack of capability to maintain optimal culture condition using the traditional technique, the life of each monolayer and its formation of e-PUK cells are limited and short, particularly as the number of passages increased even without enzymatic treatment. The traditional method is also not used as a closed system during the replacement of fresh medium or during the cell transfer, and thus requires human intervention and involves a great deal of laborious manipulations; and above all is unable to control and maintain the optimal condition of each flask during each passage of operation. Therefore it is not practical as a production method. In this disclosure a bioreactor is used and capable of controlling the condition so that the single one monolayer cells produced from the single passage lasts for a long life and used for the entire production. In the following figures the new method is illustrated.

FIG. 2 is a schematic diagram showing an automatic T75 flask bioreactor used to produce keratinocyte (e-PUK) cells continuously in a semi-continuous mode for greatly expanded time. The automatic T75 flask bioreactor is to modify a regular T-75 culture flask by adding two ports on the cap of flask allowing to connect to external gas mixture and fresh medium supply lines; and to apply an intelligent rocking device which is a modified rocker with greatly increased flexibility in angle range and rocking rate with built-in intelligence to perform filling, semi-continuous culturing, medium exchange, oxygen tension change, emptying and harvesting by applying the previously established control schemes. FIG. 2 shows that the flask bioreactor 2a is initially seeded with the primary cells 1 obtained from tissue of a biopsy using a traditional method, then allows the seeded cells to grow and reach over 80% confluency, then starts to operate as a semi-continuous or continuous culture by feeding fresh medium or replacing the spent medium with fresh medium 3a in a control manner to retain optimal nutrient condition; and to also control optimal oxygen tension 4a to best maintain the life of monolayer; and also remove the fragile floating progenitor stem-like small cells 5 (referred to as e-PUK cells) along with the medium replacement. The frequency of medium replacement or e-PUK cells harvesting is adjusted according to the glucose consumption rate (as an indicator). In each interval between two medium replacement cycles, the glucose consumption is determined by the glucose assays using some glucose meter or glucose analysis. The next cycle time is then calculated using Equation 1 or 2 established above to maintain the glucose concentration in the culture medium close to but not below the minimum critical level for the best economical interest. This calculated cycle time is then used for the next medium replacement and cell harvesting. The low oxygen tension is also applied to benefit the production of many stem cells according to literature. The e-PUK cells 5 are ideally harvested directly from the bioreactor for immediate clinical or research use. However, the e-PUK cells are also easily harvested by letting the cells attach to the surface of another flask or like and grow to confluency and then be harvested and cryopreserved using a traditional method for later use as keratinocyte cells 6. Even though the cells in the latter case are through one time enzymatic treatment, the cells are far less damaged by enzymatic treatment obtained currently by traditional culture method which requires far greater number of passages of enzymatic treatments.

In FIG. 3 is further illustrated an integrated automatic T-flask bioreactor using a novel rocker and a modified T-75 flask to perform the process shown above in FIG. 2. The system comprises an integrated rocker 8 with digital panel 9 mounted with a single T-flask (such as Corning T75) 10, a gas mixture system 11 to feed the gas mixture through inlet port 12 and exit from port 13 through the dispensing system 14 comprising several pinch valves to external designated containers 19, 20 with outlet air filters inside of a CO2 incubator or a refrigerator 15, a pumping system 16 to pump fluid (medium, seed etc.) from the storage containers 17 through inlet port 12 for a fixed volume of 30 ml after the platform 18/vessels 10 return to the horizontal position, the content of flask 10 is programmed based upon the glucose consumption rate to empty and/or harvest by tilting the platform 18 to an angle and through the dispensing system 14 to direct the harvest line to external designated containers such as T-flask 19 or bottle 20 inside of a CO2 incubator or a refrigerator 15, the lid 21 covers the flask 10 and sits on platform 18, the temperature of the enclosure is controlled with a hot air heater 22, a portable image monitoring device 23 such as Lonza's CytoSmart or a biomass/cell monitoring device such as Capacitance RF impedance sensor placed on the platform remotely monitored, recorded and processed by a PC 25, also an analytic instrument 24 such as Biochemical analyzer or Cell Counter or Near Infrared analyzer or like is integrated with the system automatically sampled from the vessel 10 and analyzed and fed the data to PC25 to process the calculation and fed to the smart rocker 8 for execution of the control variables such as next cycle time or feed rate.

The process started from a single monolayer growth of primary epithelial cells isolated from adult human epidermis or oral mucosa or ureters at T-flask 10 with intermittent replacement of standard volume of fresh medium (e.g. 15 ml in T75 flask) every two to three days from the fresh medium bottle 17 automatically. The spent medium was discarded or saved for analysis. As the monolayer reached greater than 80% growth, the T-flasks were replaced with 2× volume of fresh medium (e.g. 30 ml in T75 flask) automatically and production of e-PUK cells began. The gas mixture was regulated to a less oxygen tension (<21% O2, e.g. 5%) through 11 and fed to the system through 12 at constant gas flow rate to the system. Initially, the monolayer in the T-flasks was cultivated at horizontal position under static condition in the device for two 24 hour cycles and the content of e-PUK cells harvested and replaced with 30 ml of fresh medium in each cycle. The initial and end samples of each cycle were analyzed for glucose concentration. Then the next cycle time was calculated by Equation (1) established above and the process proceeded. The process continued in the same manner for substantially extended time (for months). Each cycle of e-PUK cells was harvested at bottle 20 for immediate soft tissue fabrication use or at another T-flask 19 in a CO₂ incubator 15. The cells collected at T-flask 19 were subsequently further attached, cultivated, harvested, cryopreserved using traditional method for later use.

### EXAMPLE 1

As the culture started to form the e-PUK cells, it followed the protocol of traditional technique to replace the medium and harvest the daughter cells in the first two days automatically using the bioreactor system. The concentration of fresh medium C₀ was 150 mg/dl, the first day cycle 1 was t₁=24 hr, dC1=36 mg/dl and the second day cycle 2 was t₂=24 hrs., dC₁=54 mg/dl, therefore dR=54/24−36/24=0.75

In order to have the concentration in the end of next cycle close to Cₘᵢₙ=100 mg/dl, the next cycle time was calculated as:

t₃=(150−100)/(0.75+54/24)=16.67 hrs.

In the end of this cycle time the medium replacement and harvest were automatically conducted.

### EXAMPLE 2

After the semi-continuous process has progressed for some time, the 1^{st} cycle 1 of the most recent 2 cycles was t₁=18 and dC₁=45, and the 2nd cycle 2 was t₂=17.65 and dC₂=52

Therefore dR=52/17.65−45/18=0.446

In order to reach the concentration in the end of the next cycle close to Cₘᵢₙ=100 mg/dl,

The next cycle time was calculated as t₃=(150−100)/(0.446+52/17.65)=14.74 hrs.

FIG. 4 further illustrates a production bioreactor using modified vessel with multi-layer of surface plates, such as Corning's Hyperflask, Cellstack or Thermo's Cell Factory or Pall's Xpansion, to perform the semi-continuous process. All of these multi-plate bioreactors were developed for safe, large-scale production of traditional 2-D cell cultures. The multi-plate structures are comprised of multiple layer of surface plates to increase cell growth surface area (up to 122,400 cm²) compared to the small surface area of 75 cm² available in the T-75 flask bioreactor shown in FIG. 3. It is up to 1632 fold increase of surface area and thus requires substantial increased quantity of the seed. The process begins with a T175 flask bioreactor 2a using the same protocol with optimal control of nutrient 3a and oxygen 4a as shown in FIG. 2 and FIG. 3 to continuously produce the e-PUK daughter cells 5 from the P-0 monolayer which are directly used to seed the production bioreactor 2b. The e-PUK cells quickly attach to the multi-plates of 2D surface carrier in the bioreactor 2b and continue the growth and production process using the same protocol applying the same optimal control of nutrient 3b and oxygen 4b as that in the seed bioreactor 2a.

During the seeding and growing process the production of e-PUK cells from the bioreactor 2b is also self-seeding to the available open surfaces along with the e-PUK cells from the seed T175 bioreactor 2a until all complete surfaces are fully occupied. Then the full production process begins. The production bioreactor 2b continues to apply the same optimal control of nutrient 3b and oxygen 4b and produce e-PUK cells 5 and keratinocyte cells 6 from the same P-1 monolayer throughout the entire production process for a greatly expanded time.

For some of commercial multi-plate vessels such as Corning's Hyperflask, Cellstack or Thermo's Cell Factory require to turn the vessel in the second dimension to complete the culture operation process. The bioreactor system comprises of a novel rocker with capability of rocking 180 degrees in one dimension and 90 degrees in the second dimension to accommodate the operation requirement. The rest of set up and operation of this production system is the same as the small system shown in FIG. 3.

As disclosed herein a novel continuous production method of producing progenitor cells of keratinocyte cells from a monolayer cell of primary cells was developed and demonstrated. The other cell lines such as melanocyte, fibroblast, endothelial cell, urethral cell, skin cell, gingival cells, tongue cells, ligament cells, and mesothelial cells which possess growth mechanism of cell inhibition and have the similar pop-up cell phenomenon under each specific optimal medium and condition as keratinocyte cells are able to apply this novel production method to continuously produce their progenitor and primary cells for a greatly expanded time without enzyme digestion for subculturing as traditional culturing methods.

While the present disclosure has been described in connection with what is considered the most practical and preferred embodiment, it is understood that this disclosure is not limited to the disclosed embodiment but is intended to cover various arrangements included within the spirit and scope of the broadest interpretations and equivalent arrangements

### REFERENCES

U.S. Pat. No. 8,835,169 Compositions, Methods and Systems for preparation of a stem cell-enriched cell population. Stephen Feinberg (2014)

Characterization of a unique technique for culturing primary adult human epithelial progenitor/“stem cells” Marcelo et al: BMC Dermatology 12:8 (2012)

Characterization of cultured epithelial cells using a novel technique not requiring enzymatic digestion for subculturing; Peramo et al: Cell Tissue Bank 14:423 (2013)

Scaling Up Stem Cells: Moving from Laboratory to Commercial Production with a Single-Use Multiplate Bioreactor. M. Egloff et al: BioProcess International, 13(8)SEP

What is claimed is:

1. A method of continuous mass production of progenitor stem or stem-like cells using a bioreactor system without requirement of enzyme digestion for subculturing, and from one single monolayer cells, said method comprising:
a. seeding a bioreactor with primary cells derived from the tissue obtained from a biopsy, a cell bank of primary cells, or progenitor cells of said primary cells directly from another seed bioreactor;
b. incubating attached cells under controlled conditions to form a monolayer of parent cells;
c. culturing in a semi-continuous or continuous mode; wherein during the culturing the nutrient and oxygen tension is maintained at a condition to enable the life of the monolayer cells to continually proliferate and produce the pop-up progenitor cells and subsequently the primary cells for a greatly expanded time; wherein enzyme digestion is not used to facilitate subculturing;
d. removing the suspended pop-up progenitor cells along with the medium replacement under controlled timely manner; and
e. harvesting the progenitor pop-up cells directly from the bioreactor for immediate clinical use, cryopreservation, or removal to seed a second larger production bioreactor or to seed a flask for traditional cell production for cell banking.

2. The method of claim 1, wherein the bioreactor is a closed system bioreactor.

3. The method of claim 1, wherein the bioreactor comprises a 2D or 3D carrier.

4. The method of claim 3, wherein the monolayer of step b forms on these carriers.

5. The method of claim 1, wherein the progenitor/stem-like cells are progenitor cells of keratinocyte, melanocyte, fibroblast, endothelial cell, urethral cell, skin cell, gingival cells, tongue cells, ligament cells, and mesothelial cells or likes.

6. The method of claim 1, wherein said bioreactor comprises multiple openings and peristaltic pumps for introducing or removing liquid with outer containers, or for gas and medium exchange, and a control mechanism.

7. The method of claim 1, wherein said control scheme for nutrient replacement of the monolayer cell comprises adjusting the medium replacement frequency (cycle time t₃) in semi-continuous mode by the equation:
t₃=(C₀−Cₘᵢₙ)/(dR+dC₂/t₂)
where t₁ and t₂ are the first and second cycle time of the most recent 2 cycles; dC₂ is the difference of concentration change of the key nutrient component represented by glucose during the second of the most recent 2 cycles; C₀ is the concentration (mg/dl) of the fresh medium; Cₘᵢₙ is the minimum concentration to be maintained in the culture; dR=dC₂/t₂−dC₁/t₁ and is the change of the key nutrient component consumption rates represented by glucose between the two previous cycles, cycle 1 & 2, where dC₁ is the same as dC₂ but for cycle 1.

8. The method of claim 6, wherein the continuous feeding rate F₃ for the next monitoring cycle is calculated by the following equation:
F₃=dR/(C₀−Cₘᵢₙ)
where dR=dC₂/t₂−dC₁/t₁ is the change of the key nutrient component consumption rates represented by glucose between the initial two previous monitoring cycles, cycle 1 & 2, as calculated in the previous semi-continuous mode, then the following cycle times are the maximum permissible time for total medium replacement in the continuous operation mode

9. The method of claim 1, wherein said removal of pop-up progenitor cells in a controlled timely manner to prevent the cells from dying and poisoning the monolayer cells is dependent upon the maximum permissible time that said progenitor cells can remain alive in suspension without attachment, and accomplished by discharging said progenitor cells along with the spent medium during the complete medium replacement in semi-continuous operation mode or intermittent total medium replacement at the maximum permissible time in continuous operation mode.
